(19) United States
(12) Patent Application Publication
(10) Pub. No.: US 2006/0142565 A1
(43) Pub. Date: Jun. 29, 2006

(54) Method of purifying tacrolimus

(76) Inventors: Vilmos Keri, Debrecen (HU); Andrea Csorvasi, Debrecen (HU); Istvan Melczer, Debrecen (HU); Angela Simon, Debrecen (HU)

Correspondence Address: KENYON & KENYON LLP, ONE BROADWAY, NEW YORK, NY, 10004, US

(21) Appl. No.: 11/317,155

(22) Filed: Dec. 22, 2005

Related U.S. Application Data
(60) Provisional application No. 60/638,628, filed on Dec. 22, 2004.
(60) Provisional application No. 60/638,815, filed on Dec. 23, 2004.

(51) Int. Cl. C07D 281/18

(52) U.S. Cl. 540/456; 540/457

(57) ABSTRACT

The invention provides pure tacrolimus and a method for purifying tacrolimus in which a loading charge of tacrolimus is placed in juxtaposition with a bed of wet sorption resin, the loading charge and bed are eluted with an eluent comprising THF, acetonitrile, or a combination thereof, water, and, optionally, at least one additional organic solvent, the heart cut of the eluent is collected, and tacrolimus is collected, the tacrolimus is further crystallized and recrystallized until obtaining a reduced level of impurities.

### RELATED APPLICATIONS

This application claims the benefits of U.S. Provisional Applications Ser. Nos. 60/638,628, filed Dec. 22, 2004, and 60/638,815, filed Dec. 23, 2004, the contents of which are incorporated herein by reference in their entirety.

The present invention relates to pure tacrolimus and to a method of purifying the macrolide tacrolimus, using sorption resins.

### BACKGROUND OF THE INVENTION

Macrolides are multi-membered lactone rings having one or more deoxy sugars as substituents. Tacrolimus (FK 506) is a macrolide antibiotic that is also an immunosuppressive agent. More potent than cyclosporin, tacrolimus reportedly has a selective inhibitory effect on T-lymphocytes.

Tacrolimus is typically obtained by fermentation. Tacrolimus, as obtained, typically contains several impurities that can be detected by various means, for example high-pressure liquid chromatography (HPLC). The presence of impurities in a pharmaceutical compound is undesirable, and health authorities in many jurisdictions, e.g. the Food and Drug Administration in the United States, have established guidelines relating to acceptable levels of impurities in pharmaceuticals. The need for, and commercial utility of, methods of reducing the level of impurities in any pharmaceutical are self-evident.

U.S. Pat. Nos.: 4,894,366, 6,576,135, 6,881,341 and 6,492,513 disclose purification processes of tacrolimus.

The inventor (Fujisawa) tablets are marketed under the name PROGRAF®. PROGRAF® tablets were analyzed and found to contain several impurities. The tablet impurity profile is summarized in table 1.

TABLE 1

| | Area percent as determined by |
|---|---|
| Impurity | HPLC |
| Total impurities | 0.51 |
| Dihydrotacrolimus | 0.07 |
| Ascomycin | 0.06 |
| RRT 0.60 | 0.12 |
| RRT 0.83 | 0.12 |
| RRT 1.45 | 0.08 |

Therefore, a need exists for tacrolimus having a higher purity than that which was achieved before, as well as a more efficient method for the purification of tacrolimus.

### SUMMARY OF THE INVENTION

In one embodiment, the present invention is directed to tacrolimus having a total impurities content of less than about 0.50 area percent, and, preferably, less than about 0.16 area percent by HPLC.

In another embodiment, the present invention is directed to tacrolimus having less than about 0.06 area percent, and, preferably, less than about 0.02 area percent by HPLC of ascomycin.

In another embodiment, the present invention is directed to tacrolimus having less than about 0.07 area percent, and, preferably, less than about 0.05 area percent by HPLC of dihydrotacrolimus.

In another embodiment, the present invention is directed to tacrolimus having less than about 0.02 area percent by HPLC of the impurity RRT 1.19.

In another embodiment, the present invention is directed to tacrolimus having less than about 0.12 area percent, and, preferably, less than about 0.02 area percent by HPLC of the impurity RRT 0.60.

In another embodiment, the present invention is directed to tacrolimus having less than about 0.12 area percent, and, preferably, less than about 0.02 area percent by HPLC of the impurity RRT 0.83.

In another embodiment, the present invention is directed to tacrolimus having less than about 0.08 area percent, and, preferably, less than about 0.02 area percent by HPLC of the impurity RRT 1.45.

In another embodiment, the present invention is directed to tacrolimus having less than about 0.06 area percent, and, preferably, less than about 0.02 area percent by HPLC of any individual impurity.

In one embodiment, the present invention provides a method for purifying tacrolimus. The method comprises providing a loading charge of tacrolimus, loading the loading charge of the tacrolimus onto a bed of sorption resin, eluting the loading charge and bed with an eluent that contains THF and/or acetonitrile and water, to obtain an effluent, collecting the main fraction of the effluent, recovering the tacrolimus from the main fraction, crystallizing the tacrolimus and further recrystallizing it. Preferably, the tacrolimus obtained in the above process has a level of impurities as described above. The tacrolimus can be from any source.

In another aspect, the present invention relates to the tacrolimus prepared according to the method described above.

### DETAILED DESCRIPTION OF THE INVENTION

As used herein, the term “ambient temperature” refers to a temperature of about 0° to about 40° C., preferably of about 10° to about 35° C.

As used herein, the term “reduced pressure” refers to a pressure of less than about 760 mm Hg.

As used herein, the term “anti-solvent” refers to a substance, normally liquid at ambient temperature, in which tacrolimus is at best sparingly soluble.

As used herein, the term “impurity” relates to any compound having a retention time that differs from that of tacrolimus by at least the detection limit of the chromatography apparatus used to determine the retention time. The different retention time may be measured, for example, by the HPLC method described herein below.

As used herein, the term “impurity RRT 1.19” relates to an impurity appearing at an RRT of about 1.19, in an HPLC chromatogram. This impurity is an isomer of tacrolimus.

As used herein, the term “impurity RRT 0.60” relates to an impurity appearing at an RRT of about 0.60, in an HPLC chromatogram.

As used herein, the term “impurity RRT 0.83” relates to an impurity appearing at an RRT of about 0.83, in an HPLC chromatogram.

As used herein, the term “impurity RRT 1.45” relates to an impurity appearing at an RRT of about 1.45, in an HPLC chromatogram.

As used herein, the terms ascomycin and dihydrotacrolimus refer to RRT0.95 and RRT1.25, respectively, which are impurities in tacrolimus, having retention times, relative to tacrolimus, of about 0.95 and 1.25 in HPLC analysis, such as the one described herein below. As used herein in connection with mixtures or combinations of liquids, the terms “volume percent” and “percent-by-volume” (vol-%) refer to a volume fraction calculated as follows (illustrated for species A):
vol-%_{A}=Wt_{A}×ρ_{A}/(Wt_{A}×ρ_{A}+Wt_{B}×ρ_{B})
where Wt_{A} and Wt_{B} are the weights in grams of species A and B, respectively, and ρ_{A} and ρ_{B} are the densities, in g/ml. of species A and B, respectively.

In one embodiment, the present invention is directed to tacrolimus having a total impurities content of less than about 0.50 area percent, and, preferably, less than about 0.16 area percent by HPLC.

In another embodiment, the present invention is directed to tacrolimus having less than about 0.06 area percent, and, preferably, less than about 0.02 area percent by HPLC of ascomycin.

In another embodiment, the present invention is directed to tacrolimus having less than about 0.07 area percent, and, preferably, less than about 0.05 area percent by HPLC of dihydrotacrolimus.

In another embodiment, the present invention is directed to tacrolimus having less than about 0.02 area percent by HPLC of the impurity RRT 1.19.

In another embodiment, the present invention is directed to tacrolimus having less than about 0.12 area percent, and, preferably, less than about 0.02 area percent by HPLC of the impurity RRT 0.60.

In another embodiment, the present invention is directed to tacrolimus having less than about 0.12 area percent, and, preferably, less than about 0.02 area percent by HPLC of the impurity RRT 0.83.

In another embodiment, the present invention is directed to tacrolimus having less than about 0.08 area percent, and, preferably, less than about 0.02 area percent by HPLC of the impurity RRT 1.45.

In another embodiment, the present invention is directed to tacrolimus having less than about 0.06 area percent, and, preferably, less than about 0.02 area percent by HPLC of any individual impurity. [00035] In one embodiment, the present invention provides a method for purifying tacrolimus, i.e. reduction of the impurities content in tacrolimus. The method comprises providing a loading charge of tacrolimus, loading the loading charge of the tacrolimus onto a bed of sorption resin, eluting the loading charge and bed with an eluent that contains THF and/or acetonitrile and water to obtain an effluent, collecting the main fraction of the effluent, recovering the tacrolimus from the main fraction, crystallizing the tacrolimus and further recrystallizing it. Preferably, the tacrolimus obtained in the above process has a level of impurities as described above. The tacrolimus can be from any source.

In the practice of the present invention, reduction or separation of impurities is mostly effected by eluting a bed of sorption resin, loaded with a loading charge of tacrolimus, with an eluent to obtain an effluent. The sorption resins useful in the practice of the present invention are well-known in the art, and are preferably cross-linked, non-ionic styrene-divinyl benzene materials, which can be chemically modified. Acrylic-type sorption resins are also known. The sorption resins have highly porous structures, having surfaces that can absorb, and then desorb, various chemical species. The absorption and desorption are influenced by the environment, for example the solvent used. In the presence of polar solvents (e.g. water) the sorption resins exhibit hydrophobic behavior. When non-polar solvents are used (e.g. hydrocarbons), the sorption resins can exhibit some polar behavior. Typically, sorption resins have a macroreticular structure, and have surface areas of at least about 300 m²/g.

Sorption resins useful in the practice of the present invention include the AMBERLITE® XAD resins available from Rohm and Haas; XAD 4, XAD 7 HP, XAD 16 HP, XAD 761, and XAD 1180, to mention just a few. Also useful are the Diaion sorption resins available from Mitsubishi; HP 10, HP 20, HP 21, HP 30, HP 40, HP 50, SP 800, SP 825, SP 850, SP 875, SP 205, SP 206, SP 207, HP1MG and HP2MG, to mention just a few. AMBERLITE® XAD 1180 is an example of a preferred sorption resin for use in the practice of the present invention. AMBERLITE® XAD 1180 is a macroreticular cross-linked aromatic polymer. It is a non-ionic, hydrophobic, cross-linked polymer which derives its adsorptive properties from its patented macroreticular structure (containing both a continuous polymer phase and a continuous pore phase), high surface area, and the aromatic nature of its surface. Surface area is 500 m²/g or higher. Porosity is 0.60 ml/ml or higher. Product data sheet of PDS 0205 A-Jan.98-1/2 gives further information about this resin.

The loading charge can be provided as a solution of the tacrolimus in an organic solvent, or in an organic solvent combined with water, or as tacrolimus-loaded loading portion that is a tacrolimus which is adsorbed onto a loading portion of sorption resin.

When, the loading charge of the tacrolimus is adsorbed onto (deposited onto) a loading portion of sorption resin prior to loading onto the bed of sorption resin, the adsorption includes preparing a solution of the tacrolimus in an organic solvent, optionally containing water and combining the solution with a portion of sorption resin and water. The sorption resin can be the same as that used to prepare the bed, or it can be a different sorption resin. The loading portion of sorption resin can be about 33 percent to about 50 percent the volume of the bed. After the adsorption of tacrolimus on the sorption resin is substantially complete, the loading charge is separated from the remaining solution. Separation can be by filtration. When the recirculating column method for making the loading charge is used, the column is simply decoupled from the recirculating system.

The organic solvent used to prepare the solution from which the loading charge is loaded or deposited is preferably selected from the group consisting of tetrahydrofuran (THF), acetone, acetonitrile (ACN), methanol, ethanol, n-butanol, n-propanol, iso-propanol, esters (e.g. ethyl acetate), and dipolar aprotic solvents, such as dimethylformamide (DMF). More preferably, the organic solvent is THF, acetone or ACN, and, most preferably, THF and ACN.

The addition of water reduces the solvent:water ratio and therefore increases the adsorption of tacrolimus on sorption resin.

The combination of the loading charge of the tacrolimus solution, loading portion of sorption resin, and water can be in any convenient vessel equipped with an agitator (e.g. a stirred tank reactor).

By way of example, the loading charge of the tacrolimus solution can be about 100 g/l, and the volume of water can be at least about five times the volume of solution. The bulk volume of the loading portion of sorption resin can be approximately equal to the volume of solution. The skilled artisan will know to optimize the proportions by routine experimentation to obtain adsorption of the tacrolimus on the loading portion of the sorption resin.

In a subsequent step of this embodiment, the now tacrolimus-loaded loading portion is juxtaposed to a prepared bed of wet sorption resin. The bed is confined in a suitable vessel. Preferably, the bed is confined within a column, preferably of circular cross-section. To prepare the bed, the desired amount of sorption resin is slurried with water or a mixture of water and a solvent (e.g. THF or ACN). A water-solvent combination is advantageous when the bed is to have a large diameter.

Separation of tacrolimus and impurities, whereby the level of impurities in the tacrolimus is reduced to provide a pure tacrolimus, is done by passing an eluent through the loading charge and subsequently through the bed of sorption resin juxtaposed thereto and in fluid communication therewith. Optionally, the eluent comprises an additional organic solvent selected from the group of solvents that are used for dissolving the tacrolimus in the first step of the process.

In the case that the loading charge is provided as a solution of the tacrolimus in an organic solvent, or in an organic solvent combined with water, the solution is injected into the prepared bed of wet sorption resin, the column is contacted with the flow of tacrolimus solution, the eluent is introduced into the stream of solution flowing through and around the loading portion of sorption resin, whereby the tacrolimus sample is gradually adsorbed onto the loading portion of sorption resin.

After the first elution, the bed may be placed in fluid communication with a second bed so that effluent from the first bed elutes through the second bed. After elution of first and second beds, the second bed may be, and, preferably, is decoupled from the first bed (i.e. fluid communication is broken) and elution is continued through the second bed alone. Optionally, the eluent is a mixture of THF and water having about 33 volume percent to 37. The eluent fractions may be collected and diluted with water, and thereafter may pass threw a third bed (column). Optionally, additional columns may be connected to the system and are diluted with additional amount of water in order to obtain a purer product. Preferably, additional amount of water is added to the last column in order to increase the adsorption of tacrolimus onto the sorption resin.

The eluent includes water and an organic solvent, such as THF, can and mixtures thereof. A preferred eluent is essentially a mixture of THF and water having about 20 volume percent to about 50 volume percent, most preferably about 31 volume percent to about 40 volume percent, THF. When an organic solvent, such as methanol, acetonitrile, acetone, or n-butanol, is used with the THF/water eluent, the THF content is less than 38 volume percent, preferably between about 4 and about 38 volume percent. Another preferred eluent is a mixture of acetonitrile and water having about 30 volume percent to about 70 volume percent, most preferably about 40 volume percent to about 65 volume percent, acetonitrile. When the eluent is a mixture of acetonitrile and water, the eluent can also include about 0.0005 to about 0.003 parts phosphoric acid to 1 part eluent.

The eluent is eluted through the loading charge and bed of sorption resin juxtaposed thereto at a rate that depends on the gross cross-sectional area of the bed (measured perpendicular to the flow of eluent). Preferably, the flow rate (relative to the cross-sectional area) is less than about 25 cm/hour, preferably less than about 15 cm/hour. Lower elution rates increase the time, but improve the separation efficiency. A preferred elution rate for increased separation efficiency is about 9 cm/hour to about 11 cm/hour.

The content and composition of the eluted fractions can be monitored by any convenient means. Detection and quantification of impurities in tacrolimus, in particular ascomycin and dihydrotacrolimus, can be carried-out by the hereinbelow described HPLC method.

Depending on, inter alia, column loading and the composition and flow rate of the eluent, the main fraction is collected, so that the final isolated product has about 0.1 area percent or less by HPLC of ascomycin.

If desired, the tacrolimus separated from impurities and therefore having a reduced level of impurities can be isolated from effluent by any conventional means (e.g. extraction, lyophilization, evaporation, addition of anti-solvent). Water, alkanes and cycloalkanes are useful anti-solvents, and others are known in the art. Isolation methods can be combined. For example anti-solvent can be combined with concentrated eluent.

A preferred method of isolation includes concentration of the main fraction at 70° C. or less, preferably 60° C. or less, preferably at pressure of 760 mm Hg or less, to about 50 percent of its initial volume, whereby concentrated tacrolimus fraction is obtained. Phosphoric acid, about 1 to about 10 ml per liter of eluent is preferably added before concentration to stabilize the tacrolimus.

Optionally, the concentrated main fraction is maintained at ambient temperature for a holding time. When a holding time is used, a preferred holding time is about 1-4 days. Water immiscible solvent such as ethyl acetate or dichloromethane, and a base, such as ammonia solution, are added to the concentrated tacrolimus fraction and the water immiscible solvent phase is separated and concentrated. The base is added until the pH is of about 9 or less.

Further reduction in impurities can be achieved by subjecting the recovered product to several additional treatments such as crystallization and recrystallization.

Crystallization of the oily residue of tacrolimus comprises dissolving the oily residue of tacrolimus in ethyl acetate and cyclohexane, adding water to induce crystallization of tacrolimus and recovering the crystallized tacrolimus. Preferably, prior to the dissolving step, the oily residue is diluted with ethyl acetate and concentrated again to oily residue. Preferably, the water is added drop-wise. Typically, the water:tacrolimus ratio is 0.015 kg to 0.3 kg water to 1 kg tacrolimus in the crystallization process.

Recrystallization of tacrolimus comprises dissolving the tacrolimus in ethyl acetate, concentrating the solution until obtaining an oily residue, dissolving the oily residue in ethyl acetate, adding cyclohexane to the solution, adding water to induce crystallization of tacrolimus and recovering the crystallized tacrolimus. Preferably, dissolution and concentration steps may be repeated. Preferably, the solution is treated with charcoal in order to remove the color and fibers. Concentration is as described above. Preferably, the obtained tacrolimus is further dried.

In the practice of the present invention the last chromatography step was carried out with three columns in series, resulted in significant reduction of ascomycin and dihydrotacrolimus, and the regulated addition of water to the solvent mixture and a regulated crystallization time resulted in significant reduction of the impurity RRT 1.19.

The purification of tacrolimus, accomplished by the method of the present invention, can be monitored by the HPLC method described hereinbelow.

In a particular embodiment, at least the levels of impurities ascomycin and dihydrotacrolimus are reduced to provide a high purity tacrolimus. The levels of other impurities are also reduced. The method includes the steps of: preparing a loading charge of tacrolimus comprising a solution of tacrolimus with or without a loading portion of a sorption resin, especially a macroreticular resin, such as AMBERLITE® XAD 1180 and Diaion HP 20; loading the loading charge to wet sorption resin, especially AMBERLITE® XAD 1180 and Diaion HP 20, that can be contained in a vessel, especially a column; eluting the loading portion and sorption resin with an eluent that is a mixture of tetrahydrofuran (THF) and water, about 20 volume percent to about 50 volume percent, especially about 31 volume percent to about 40 volume percent THF, or a mixture of acetonitrile (ACN) and water, about 30 volume percent to about 70 volume percent and most especially about 40 volume percent to about 65 volume percent acetonitrile; collecting at least a main fraction (heart cut) of eluent that contains more than about 60 percent, preferably between about 60 percent and about 90 percent of the initial tacrolimus, (depending on the initial purity) and, optionally, isolating tacrolimus having reduced impurities from the main fraction by, for example, concentrating the main fraction(s), for example at reduced pressure in the presence of an acid, and, optionally, recovering the product so obtained. The obtained product is further crystallized and recrystallized as described above.

Preferably, the obtained tacrolimus has less than 0.50 area percent, most preferably, less than 0.16 area percent by HPLC of total impurities content.

Preferably, the obtained tacrolimus has less than about 0.06 area percent, most preferably, less than about 0.02 area percent by HPLC of ascomycin.

Preferably, the obtained tacrolimus has less than about 0.07 area percent, most preferably, less than about 0.05 area percent by HPLC of dihydrotacrolimus.

Preferably, the obtained tacrolimus has less than about 0.02 area percent by HPLC of the impurity RRT 1.19.

Preferably, the obtained tacrolimus has less than about 0.12 area percent, most preferably, less than about 0.02 area percent by HPLC of the impurity RRT 0.60.

Preferably, the obtained tacrolimus has less than about 0.12 area percent, most preferably, less than about 0.02 area percent by HPLC of the impurity RRT 0.83.

Preferably, the obtained tacrolimus has less than about 0.08 area percent, most preferably, less than about 0.02 area percent by HPLC of the impurity RRT 1.45.

Preferably, the obtained tacrolimus has less than about 0.06 area percent, most preferably, less than about 0.02 area percent by HPLC of any individual impurity.

The present invention provides the tacrolimus obtained by the above process.

### Chromatographic Conditions Used for Examples

Column: ZORBAX SB-C18 75×4.6 mm; 3.5 μm
Pre-column: SymmetryShield RP18 3.9×20 mm; 5 μm
Eluent: A: Measure 200 ml of acetonitrile into a 2000 ml volumetric flask, then dilute to volume with distilled water to 2000 ml total volume. Then, add 100 μl of 50 percent acetic acid. B: Add 100 μl of 50 percent acetic acid to 2000 ml of acetonitrile.

Table of Gradients

| Time | Eluent “A” | Eluent “B” | Flow rate |
|---|---|---|---|
| (min) | (w/w %) | (w/w %) | (ml/min) |
| 0 | 60 | 40 | 2.3 |
| 15 | 55 | 45 | 2.3 |
| 25 | 30 | 70 | 1.8 |
| 25.1 | 60 | 40 | 1.8 |
| 27 | 60 | 40 | 1.8 |

Flow rate: 2.3 ml/min
Detection wavelength: 210 nm
Injected volume: 20 μl
Sample's solvent: acetonitrile
Temp. of column unit: 60° C.
Analysis time: 27 min
Retention time of tacrolimus: appr. 14 min
Detection Limit: less than 0.01 area percent
Quantification Limit: less than 0.02 area percent.

Retention times of impurities ascomycin (RRT 0.95), dihydrotacrolimus (RRT 1.25) and the impurity RRT 1.19 are relative to tacrolimus and expressed as an area percent relative to the area of all peaks in the chromatogram.

The detection and quantification limits for typical HPLC equipment available at present are less than 0.01 area percent and less than 0.02 area percent, respectively.

### EXAMPLES

The following non-limiting examples are merely illustrative of the preferred embodiments of the present invention, and are not to be construed as limiting the invention, the scope of which is defined by the appended claims.

### Example 1

A tacrolimus starting material was purified by chromatography and several crystallization steps. The purity analysis was conducted using the analytical HPLC method described above under “Chromatographic conditions used for examples.” The starting material contained 0.16 area percent ascomycin, 1.56 area percent of the impurity RRT 1.19, and 0.46 area percent dihydrotacrolimus. An assay of the starting substance gave a purity of 95 percent by mass. Following purification with the method of the invention, the final product contained 0.02 area percent ascomycin, 0.02 area percent of the impurity RRT 1.19, and 0.05 area percent dihydrotacrolimus. The amount of any other impurity present was no more than 0.02 area percent, and the purity of the tacrolimus obtained with the method of the invention was 99.84 area percent.

Chromatography Step

AMBERLITE® XAD 1180 sorption resin was used for the chromatography. Three chromatography columns (40 cm diameter, 1 m column height, and ca. 100 liters wet sorption resin) were prepared. The tacrolimus starting material in an amount of 3812 g, where 3623 g was active substance, was dissolved in 30 liters of acetone. The resin AMBERLITE® XAD 1180 in an amount of 33 liters was added to the tacrolimus solution. Water in an amount of 180 liters was slowly added, with agitation to the tacrolimus solution:resin mixture. When the addition of water was complete, the loading charge of sorption resin was collected by filtration.

The collected loading charge was loaded as a layer on the top of the bed of wet sorption resin. The total resin volume was ca. 100 liters. The column was first eluted with ca. 700 liters of eluent of tetrahydrofuran/water (34 vol-% THF). After the first elution, a second column was connected to the first column. The elution was continued with ca. 1400 liters of eluent of THF/water (34 vol-% THF). The first column was disconnected from the second column, and the elution was continued with ca. 1200 liters of eluent of THF/water (34 vol-% THF). Fractions having a volume of 20 liters each were collected. Water in an amount of 0.5 liter was mixed with each fraction, providing diluted fractions. The diluted fractions were passed through a third column, and tacrolimus was adsorbed on the resin of the third column. The tacrolimus was eluted from the third column with ca. 1800 liters of the THF/water (34 vol-% THF) eluent. Fractions each having a volume of 20 liters were collected, and several fractions were analyzed using the HPLC method described above in “Chromatographic conditions used for examples.”

Appropriate fractions were then combined. However, it should be noted that, prior to the combination of the fractions, preliminary fractions may be combined, e.g., 10 ml from each appropriate fraction, and analyzed with HPLC analysis. If the HPLC analysis of the preliminary combination results in higher than a 0.02 area percent ascomycin concentration and/or higher than a 0.04 area percent dihydrotacrolimus concentration, the number of combined fractions should be modified to provide the desired high purity, as obtaining the desires high purity during the combination of the fractions will provide a high purity final yield.

The combined main fraction (ca. 500 liters) was mixed with 100 ml of 85 percent phosphoric acid, and concentrated at reduced pressure to a volume of about 200 liters. The concentrate was cooled to ambient temperature, and 50 liters of water, 100 liters of ethyl acetate, and 200 ml of concentrated ammonia solution were added to the concentrate. The ethyl acetate phase (ca. 75 liters) was separated, and concentrated under reduced pressure to oily residue.

Crystallization of Main Fraction

The oily residue was diluted with 10 liters of ethyl acetate, and concentrated again to an oily residue under reduced pressure. The heating temperature was ca. 60° C., and the estimated boiling temperature was 20-40° C. The dilution-concentration step was repeated twice.

The solid content of oily residue was established by evaporation of a small amount of sample under reduced pressure, resulting in a solids content of 1329 g for the oily residue. The oily residue was diluted with ethyl acetate to ca. 2525 g, and 7970 ml cyclohexane was added to the solution. The temperature was maintained at 25° C. using a temperature circulator.

Water was added rapidly to the solution in an amount of 10.6 ml. Water in an amount of 18.6 ml was added to the solution for 3 hours, initiating crystallization. After stirring for 45 minutes, the crystals were filtered, and washed with 1600 ml of cyclohexane. The washed crystals were dried at 70° C. for 12 hours, providing a mass of dried crystals of 1250 g.

Recrystallization

Tacrolimus in an amount of 1250 g was dissolved in 7.5 liters of ethyl acetate. The solution was concentrated to an oily residue under reduced pressure. The dissolution-concentration step was repeated twice. The oily residue was dissolved in 3750 ml ethyl acetate, and treated with 12.5 g of charcoal. The charcoal treatment was carried out at 30° C. for 30 minutes. The suspension was filtered, and the filter cake was washed with 125 ml ethyl acetate. The filtered solution was concentrated under reduced pressure, and diluted with ethyl acetate to 2375 g.

Cyclohexane in an amount of 6.25 liters was added to the tacrolimus solution for 1.5 hours. Water in an amount of 27.5 ml was added to the solution for 2 hours. Water in an amount of 246 ml was added to the solution for 2 hours, initiating crystallization.

The suspension was cooled to 8° C., and cyclohexane in an amount of 1.25 liters was added to the suspension for 1 hour at 8° C. Then, the suspension was stirred at 8° C. for 12 hours. The crystals were filtered, and suspended twice with cyclohexane. The volume of cyclohexane used for the suspensions was 2.5 liters.

Drying was carried out under reduced pressure at 40° C. for 12 hours, and at ca. 25° C. for 24 hours. A nitrogen inlet was used during the whole drying process.

The crystallization steps efficiently reduce the impurity RRT 1.19 content of the product. The mass of the final product was 1180 g. The purity of the obtained tacrolimus was 99.84 area percent by HPLC, i.e.: total impurity content of 0.16 area percent by HPLC. The final product contained: 0.02 area percent by HPLC of ascomycin, 0.05 area percent by HPLC of dihydrotacrolimus and 0.02 area percent by HPLC of the impurity RRT 1.19, 0.02 area percent by HPLC of the RRT 0.83, less than 0.02 area percent by HPLC of the RRT 0.60, less than 0.02 area percent by HPLC of the RRT 1.45, 0.02 area percent by HPLC of the RRT 0.25 and less than 0.02 area percent by HPLC of any individual impurity. The impurity profile is summarized in table 2.

TABLE 2

| Impurity | Area percent as determined by HPLC |
|---|---|
| Total impurities | 0.16 |
| Dihydrotacrolimus | 0.05 |
| Ascomycin | 0.02 |
| RRT 0.60 | <0.02 |
| RRT 0.83 | 0.02 |
| RRT 1.45 | <0.02 |
| RRT 1.19 | 0.02 |
| RRT 0.25 | 0.02 |
| Any individual impurity | <0.02 |

If, following crystallization, the concentration of the impurity RRT 1.19 in the final product is greater than desired, one or more additional crystallization steps may be performed to remove the impurity.

What is claimed is:

1. Tacrolimus having a total impurities content of less than about 0.50 area percent by HPLC.

2. The tacrolimus of claim 1, having less than about 0.16 area percent by HPLC of total impurities content.

3. Tacrolimus having less than about 0.06 area percent by HPLC of ascomycin.

4. The tacrolimus of claim 3, having less than about 0.02 area percent by HPLC of ascomycin.

5. Tacrolimus having less than about 0.07 area percent by HPLC of dihydrotacrolimus.

6. The tacrolimus of claim 5, having less than about 0.05 area percent by HPLC of dihydrotacrolimus.

7. Tacrolimus having less than about 0.02 area percent by HPLC of the impurity RRT 1.19.

8. Tacrolimus having less than about 0.12 area percent by HPLC of the impurity RRT 0.60.

9. The tacrolimus of claim 8, having less than about 0.02 area percent by HPLC of the impurity RRT 0.60.

10. Tacrolimus having less than about 0.12 area percent by HPLC of the impurity RRT 0.83.

11. The tacrolimus of claim 10, having less than about 0.02 area percent by HPLC of the impurity RRT 0.83.

12. Tacrolimus having less than about 0.08 area percent by HPLC of the impurity RRT 1.45.

13. The tacrolimus of claim 12, having less than about 0.02 area percent by HPLC of the impurity RRT 1.45.

14. Tacrolimus having less than about 0.06 area percent by HPLC of any individual impurity.

15. The tacrolimus of claim 14, having less than about 0.02 area percent by HPLC of any individual impurity.

16. A process for purifying tacrolimus comprising:
a) providing a loading charge of tacrolimus;
b) loading the loading charge of the tacrolimus onto a bed of sorption resin;
c) eluting the loading charge and bed with an eluent comprising THF, acetonitrile, or a combination thereof, and water to obtain an effluent;
d) collecting the main fraction of the effluent;
e) recovering the tacrolimus from the main fraction;
f) crystallizing the tacrolimus; and
g) recrystallizing the tacrolimus.

17. The process of claim 16, wherein the sorption resin is AMBERLITE® XAD 1180.

18. The process of claim 16, wherein the loading charge is a solution of the tacrolimus in an organic solvent, or in an organic solvent combined with water.

19. The process of claim 16, wherein the loading charge is tacrolimus which is adsorbed onto a loading portion of sorption resin.

20. The process of claim 19, wherein the adsorption includes preparing a solution of tacrolimus in an organic solvent, combining the solution with a portion of sorption resin and water, and separating the adsorbed loading charge from the remaining solution.

21. The process of claim 20, wherein the separation is by filtration.

22. The process of claim 20, wherein the organic solvent is selected from the group consisting of: tetrahydrofuran (THF), acetone, acetonitrile (ACN), methanol, ethanol, n-butanol, n-propanol, iso-propanol, esters (e.g. ethyl acetate), and dipolar aprotic solvents, such as dimethylformamide (DMF).

23. The process of claim 22, wherein the organic solvent is THF, acetone or acetonitrile.

24. The process of claim 16, wherein the eluent in step c) contains at least one organic solvent.

25. The process of claim 16, wherein the bed of sorption resin is confined within a column.

26. The process of claim 16, wherein prior to step d) the bed is placed in fluid communication with a second bed of sorption resin.

27. The process of claim 26, wherein the second bed is decoupled from the first bed.

28. The process of claim 26, further comprising connecting additional beds of sorption resin to the system.

29. The process of claim 28, wherein additional amount of water is added to the last column.

30. The process of claim 16, wherein the isolation in step e) includes concentration of the main fraction in the presence of phosphoric acid, at a temperature of about 70° C. or less, at a pressure of about 760 mm Hg or less, adding water immiscible solvent and a base, separating the water immiscible solvent phase and concentrating it.

31. The process of claim 30, wherein the water immiscible solvent is ethyl acetate or dichloromethane.

32. The process of claim 30, wherein the base is ammonia solution.

33. The process of claim 16, wherein the crystallization in step f) comprises dissolving an oily residue of tacrolimus in ethyl acetate and cyclohexane, adding water to induce crystallization of tacrolimus and recovering the crystallized tacrolimus.

34. The process of claim 33, wherein prior to the dissolving step, the oily residue is diluted with ethyl acetate and concentrated again to oily residue.

35. The process of claim 16, wherein the recrystallization in step g) comprises dissolving the tacrolimus in ethyl acetate, concentrating the solution until obtaining an oily residue, dissolving the oily residue in ethyl acetate, adding cyclohexane to the solution, adding water to induce crystallization of tacrolimus and recovering the tacrolimus.

36. Tacrolimus obtained by the process of claim 16.
